(19) United States
(12) Patent Application Publication
(10) Pub. No.: US 2009/0203145 A1
(43) Pub. Date: Aug. 13, 2009

(54) METHODS FOR ANALYSIS OF VITAMINS

(76) Inventor: Min Huang, Sun Prairie, WI (US)

Correspondence Address: MCDERMOTT, WILL & EMERY, 11682 EL CAMINO REAL, SUITE 400, SAN DIEGO, CA, 92130-2047, US

(21) Appl. No.: 12/027,917

(22) Filed: Feb. 7, 2008

(51) Int. Cl. G01N 33/04

(52) U.S. Cl. 436/22

(57) ABSTRACT

The invention relates to methods for determining the level of B vitamins in a test sample. The methods can include the steps of obtaining a water soluble vitamin fraction by extraction in acidic aqueous solution of a test sample, wherein the vitamin fraction comprises one or more B vitamins having at least a predetermined minimum concentration; adding a vitamin standard comprising one or more B vitamins of known quantity to a portion of the vitamin fraction of the test sample, wherein said vitamins in the vitamin standard comprise an isotope label and wherein the vitamin standard is added in an amount sufficient to determine the level of the corresponding vitamins in the test sample; and determining the level of one or more vitamins in the test sample corresponding to one or more of the vitamins in the vitamin standard using mass spectrometry.

### BACKGROUND OF THE INVENTION

The present invention relates generally to nutritional analysis and more specifically to vitamin analysis.

Deficiencies of various nutrients still occur in all societies, including “developed” countries, and some are becoming more frequent. There are common deficiencies seen in infants and toddlers. For example, breast milk contains only small amounts of vitamin K, vitamin D and iron. Therefore, vitamin K supplementation is recommended for infants fed exclusively with breast milk to prevent early haemorrhagic disease and the later rare, but often fatal, intracranial bleeding. Vitamin D deficiency is unlikely if the mother had adequate vitamin D intakes during pregnancy, either from sun or supplements, and the baby was exposed to sunlight. However, it is difficult to be sure of all these factors and a safer policy is to give vitamin D supplements to all breast fed infants. Iron is generally not a problem initially. The new born baby does not need as many red blood cells as it does in utero. As the circulating red cells are broken down, the iron in the released hemoglobin is stored and gradually reused as the infant grows. A supply of available iron in the diet is necessary from about the age of 6 months. Other nutrient deficiencies reported include thiamin, pyridoxine, B12 and zinc.

If an infant takes sufficient amounts of a properly prepared infant formula, there will be no deficiencies of either macro or micronutrients since infant formulas have been designed to meet the full nutritional requirements of a young infant. Their composition is closely regulated by law and the enforcement agencies. However, ‘feeding accidents’ have occurred very occasionally because, during manufacture or preparation, a nutrient has been omitted or destroyed. For example, deficiencies of pyridoxine can lead to convulsions, deficiencies of chloride can cause metabolic upset, and thiamine deficiencies can result in some deaths. Iron deficiency anemia is associated with alterations of immunological, gut and mental function.

The B vitamins are a group of nutrients that play an important role in human health, including thiamine (B1), riboflavin (B2), niacin (B3), pantothenic acid (B5), and pyridoxine (B6), 4-aminobenzoic acid (PABA or B10), and the like. Although they are naturally present in some foods, deficiency of B vitamins in the human diet does occur and can cause serious health issues. Supplementing foods with B vitamins is, of course, a common solution. Therefore, measurement of B vitamins in foods, particularly infant formula, is often beneficial or even required.

The often low concentrations of vitamins in foods can make measuring the level of vitamins in various foods difficult. For example, the low concentration of B vitamins in infant formula makes it difficult to be measured by traditional measurement techniques such as HPLC. Currently, microbiological methods for measuring B vitamins are widely used at laboratories. This method is based on the growth of specific bacteria in the presence of a specific B vitamin and can only be measured one analyte per assay. The analysis process may take more than 30 hours. Poor reproducibility is another disadvantage. It is not unusual for a bacterial response to vary with different compounds and show low specificity.

Thus, there exists a need to provide rapid, efficient and quantitative methods to measure nutrients, in particular vitamins, in samples. The present invention satisfies this need and provides related advantages as well.

### SUMMARY OF INVENTION

The invention relates to methods for determining the level of B vitamins in a test sample. The methods can include the steps of obtaining a water soluble vitamin fraction by extraction in acidic aqueous solution of a test sample, wherein the vitamin fraction comprises one or more B vitamins having at least a predetermined minimum concentration; adding a vitamin standard comprising one or more B vitamins of known quantity to a portion of the vitamin fraction of the test sample, wherein the vitamins in the vitamin standard comprise an isotope label and wherein the vitamin standard is added in an amount sufficient to determine the level of the corresponding vitamins in the test sample; and determining the level of one or more vitamins in the test sample corresponding to one or more of the vitamins in the vitamin standard using mass spectrometry.

### BRIEF DESCRIPTION OF THE DRAWINGS

FIG. 1 shows the chemical structures of various B vitamins.

FIGS. 2A-2G show mass spectrometry analysis of various B vitamins. Measurements were made under positive ion mode. The fragmentation ions of the various B vitamin analytes are shown. FIG. 2A, B₁; FIG. 2B, B₂; FIG. 2C, B₃/nicotinic acid; FIG. 2D, B₃/nicotinamide; FIG. 2E, B₅/pantothenic acid; FIG. 2F, pyridoxine; FIG. 2G, PABA (4-aminobenzoic acid, B₁₀).

FIG. 3 shows an exemplary chromatogram of high performance liquid chromatography tandem mass spectrometry (LC/MS/MS) analysis of B vitamins with overlapping peaks. While the peaks overlap there is no spectral interference on each signal.

FIG. 4 shows a plot of the analyte area over internal standard area versus analyte concentration over internal standard concentration for the analysis of PABA (4-aminobenzoic acid, B₁₀).

### DETAILED DESCRIPTION OF THE INVENTION

The present invention relates to methods for rapid, accurate and cost effective analysis of trace nutrients or micronutrients such as vitamins, in particular B vitamins. A liquid chromatograph/mass spectrometry/mass spectrometry (LC/MS/MS) method has been developed that provides accurate analysis with less interference than traditional methods, is sufficiently sensitive for analysis of low concentrations of analytes in complex samples and is therefore suitable for food analysis. The method is rapid with simple sample preparation and allows the simultaneous measurement of multiple analytes.

One well known method for analysis of B vitamins utilizes microbiological methods. However, the microbiological methods have the disadvantage of being a long analytical process, are less selective and exhibit poor reproducibility. The methods of the invention are advantageous over previously described methods in that they are rapid, selective for specific forms of vitamins, are highly reproducible, and allow accurate analysis of complex samples at lower costs than previously described methods.

As disclosed herein, a method for analysis of B vitamins, including simultaneous measurement of multiple B vitamins in food samples such as milk products or infant formulas by LC/MS/MS has been developed (see Examples). Sample preparation is simple and includes extraction by acidic solvent, protein precipitation, and filtration. The method, which utilizes correction by isotope labeled internal standard, was validated with a standard from the National Institute of Standards and Technology (NIST) and showed good agreement with the certified values for the analytes. The accuracy was also demonstrated by spike recovery that was typically found between 100±6% for various matrices evaluated (see Example I). Although exemplified with B vitamins, it is understood that the methods of the invention can be applied to other vitamins or micronutrients.

In one embodiment, the invention provides a method for determining the level of trace nutrients or micronutrients such as vitamins in a test sample. The method can include the steps of obtaining a vitamin fraction of a test sample; adding a vitamin standard comprising one or more vitamins and which can contain a plurality of vitamins of known quantity to a portion of the vitamin fraction of the test sample, wherein the vitamin standard is added in an amount sufficient to determine the level of the corresponding vitamins in the test sample and wherein the vitamins in the vitamin standard comprise an isotope label; and determining the level of vitamins in the test sample corresponding to one or more of the vitamins in the vitamin standard.

As used herein, a “vitamin fraction” refers to a fraction of a sample that contains one or more vitamins desired to be analyzed. It is understood that a “vitamin fraction” does not need to include all vitamins from a sample but only those that are desired to be analyzed. Any suitable method for obtaining a vitamin fraction from a sample can be utilized, as desired.

As used herein, a “vitamin standard” refers to a standard containing one or more vitamins of known absolute or relative quantity to which corresponding vitamins from a sample can be compared for quantification. Although the vitamin standard can contain a single vitamin, generally a vitamin standard contains a plurality of vitamin standards, that is, two or more vitamin standards, which allows parallel analysis of multiple analytes from a single sample.

As used herein an “isotope label” refers to a stable isotope that can be incorporated into a reference molecule so that it is differentially labeled relative to a sample molecule. As used herein, it is understood that an isotope label, or an isotopically labeled molecule, can refer to a single differentially isotopically labeled atom or multiple atoms, as desired. A particularly useful stable isotope is deuterium, which can be readily distinguished using mass spectrometry as a heavy form relative to naturally occurring hydrogen in a sample molecule. Any of a number of isotopic atoms can be incorporated into the isotope label so long as the heavy form can be distinguished the light form of a naturally occurring molecule using mass spectrometry, for example, ¹³C, ¹⁵N, ¹⁷O, ¹⁸O or ³⁴S, depending on the atoms of the sample molecule desired to be analyzed. Particularly useful isotopes for use with B vitamin standards include ²H, ¹³C and ¹⁵N. The use of an isotope label allows analysis of a chemically identical, but isotopically distinct, standard molecule for comparison to a corresponding molecule in a sample. Such isotopic labeling is particularly suitable for mass spectrometry (MS) analysis. Thus, it is understood that the standard molecule will include a sufficient number of differentially isotopically labeled atoms for MS analysis and comparison to a sample molecule. Thus, the isotope label has at least one, and can contain 2, 3, 4, 5, 6 or more heavy atoms, as desired. Generally, an isotope label has at least a 2 mass unit differential label relative to the corresponding molecule in a sample.

In using an isotope label, differential isotopes can be incorporated into a standard molecule, which can be used to compare a known amount of a standard labeled molecule having a differentially labeled isotope from that of a sample molecule, as described in the Examples. Thus, a standard molecule having a differential isotope label can be added in a known amount and at a known concentration and analyzed in the same MS analysis or under similar conditions in a parallel MS analysis. A specific, calibrated standard can be added with known absolute amounts to determine an absolute quantity of the sample molecules. In addition, the standards can be added so that quantification relative to the standard is performed, if desired. One skilled in the art will readily understand appropriate methods for adding a standard for quantifying an analyte in a test sample.

By utilizing an isotope label, the level of vitamins in the test sample can be determined by mass spectrometry (MS). A variety of mass spectrometry systems can be employed in the methods of the invention for identifying and/or quantifying a sample molecule such as a micronutrient or vitamin. Mass analyzers with high mass accuracy, high sensitivity and high resolution include, but are not limited to, ion trap, triple quadrupole, and time-of-flight, quadrupole time-of-flight mass spectrometers and Fourier transform ion cyclotron mass analyzers (FT-ICR-MS). Mass spectrometers can be equipped with matrix-assisted laser desorption (MALDI) and electrospray ionization (ESI) ion sources, although other methods of ionization can also be used. In ion trap MS, analytes are ionized by ESI or MALDI and then put into an ion trap. Trapped ions can then be separately analyzed by MS upon selective release from the ion trap. Fragments can also be generated in the ion trap and analyzed. Sample molecules can be analyzed, for example, by single stage mass spectrometry with a MALDI-TOF or ESI-TOF system. Methods of mass spectrometry analysis are well known to those skilled in the art (see, for example, Yates, J. Mass Spect. 33:1-19 (1998); Aebersold and Goodlett, Chem. Rev. 101:269-295 (2001)). For high resolution analysis, liquid chromatography ESI-MS/MS or automated LC-MS/MS, which utilizes capillary reverse phase chromatography as the separation method, can be used (Yates et al., Methods Mol. Biol. 112:553-569 (1999)). Data dependent collision-induced dissociation (CID) with dynamic exclusion can also be used as the mass spectrometric method (Goodlett, et al., Anal. Chem. 72:1112-1118 (2000)).

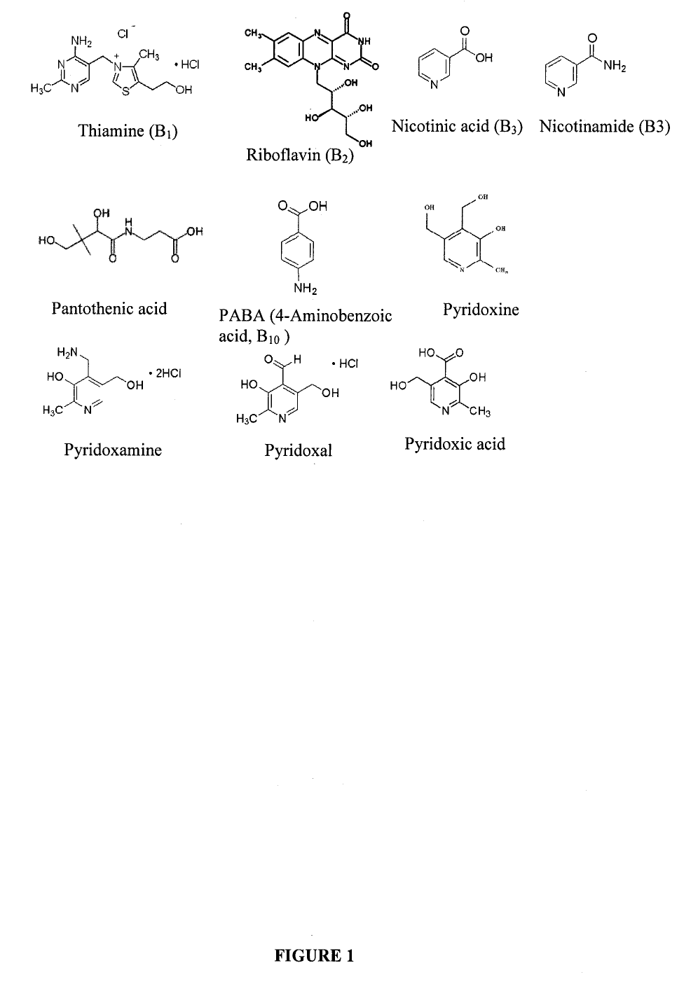

The methods of the invention are particularly useful for analyzing B vitamins using a B vitamin standard. For example, the methods can be used to analyze one or more B vitamins such as vitamin B₁, vitamin B₂, vitamin B₃, vitamin B₅, vitamin B₆ and p-aminobenzoic acid (PABA). The structures of exemplary B vitamins are shown in FIG. 1. Additional vitamins or micronutrients suitable for analysis include, but are not limited to, biotin, folic acid, vitamin B₁₂, and the like. It is understood that these and other vitamins or micronutrients can similarly be analyzed by the methods disclosed herein, as desired, so long as appropriate isotopically labeled standards can be obtained or synthesized.

In one embodiment, the invention relates to a method for determining the level of B vitamins in a test sample. The method can include the steps of obtaining a water soluble vitamin fraction of a test sample, wherein the vitamin fraction comprises one or more B vitamins having at least a predetermined minimum concentration; adding a vitamin standard comprising one or more B vitamins of known quantity to a portion of the vitamin fraction of the test sample, wherein the vitamins in the vitamin standard comprise an isotope label and wherein the vitamin standard is added in an amount sufficient to determine the level of the corresponding vitamins in the test sample; and determining the level of one or more vitamins in the test sample corresponding to one or more of the vitamins in the vitamin standard using mass spectrometry.

As used herein, a “predetermined minimal concentration” refers to a concentration of analyte such as a B vitamin or other vitamins or micronutrients determined in advance to be sufficient for detection and quantitation in the method of the invention. Generally, the minimum concentration sufficient for detection of an analyte is a concentration of analyte at the limit of detection (LOD) of the assay method being used, whereas the minimal concentration sufficient for quantitation of an analyte is the limit of quantitation (LOQ). The LOD is the lowest concentration of an analyte in a sample that can be detected, not quantitated, whereas the LOQ is the lowest concentration of an analyte in a sample that can be determined with acceptable precision and accuracy under a given set of operational conditions for the method. For example, in the case of mass spectrometry, a particular instrument under a given set of assay conditions will have an LOD and LOQ for a particular analyte, and the LOD and LOQ are known or can be readily determined using routine methods known to those skilled in the art. Thus, when a particular analyte is to be assayed and quantified in a method of the invention, the LOQ for that analyte for the instrument and under the conditions being employed in the assay are determined. The test sample is processed to obtain a water soluble vitamin fraction, as disclosed herein, and the vitamin fraction is tested to determine if a particular analyte is at a concentration at or above the LOQ for the analyte under the assay conditions being employed. Such a vitamin fraction containing a desired analyte at a concentration at or above the LOQ for the analyte under the assay conditions being employed is considered to have at least a predetermined minimum concentration of analyte suitable for a method of the invention. Once a procedure for processing the a representative sample has been determined to provide at least a predetermined minimum concentration for a desired analyte, the procedure can be repeated with similar types of test samples to determine the level of a desired analyte in the sample, as is well known to those skilled in the art.

It is well understood by those skilled in the art how to develop an extraction process for a particular test sample to achieve a predetermined minimum concentration in a vitamin fraction extracted from the test sample. In general, an extraction procedure is developed and used to process the test sample such that the vitamin fraction contains an analyte having at least a predetermined minimum concentration of a desired analyte using simple extraction procedures, without the need to further concentrate the vitamin fraction to achieve a predetermined minimum concentration of a particular analyte (see Examples). However, it is understood by those skilled in the art that, if needed, further processing and concentration of the vitamin fraction can be performed, as needed to achieve a predetermined minimum concentration.

As disclosed herein, methods for obtaining a water soluble vitamin fraction from a test sample are utilized. As discussed above, the vitamin fraction contains at least a predetermined minimum concentration of a desired analyte. It is understood by those skilled in the art that, in addition to a sample having at least a predetermined minimum concentration for the particular assay conditions being employed, an extraction procedure can result in an analyte concentration that is higher than the linear range of detection for that analyte, which is generally required for quantitative analysis. In such a case, it is well understood by those skilled in the art that such a vitamin fraction can be diluted in the appropriate buffer to achieve an analyte concentration within the linear range of the assay being employed.

As disclosed herein, a vitamin fraction is obtained containing one or more vitamins or other analytes where the one or more analytes has at least a predetermined minimum concentration. As discussed above, the predetermined minimum concentration for a particular analyte will depend on the conditions of the assay being employed, for example, type of instrument, sensitivity and settings on the instrument, and the like. Furthermore, it is understood that the predetermined minimum concentration will vary with the particular analyte. For example, in mass spectrometry, the ability to detect and quantify an analyte will depend on the sensitivity of the instrument and the ionization potential of the particular analyte. Different analytes will have different ionization potentials, and therefore the predetermined minimum concentration for a particular analyte will vary with the analyte and depend on the ionization potential. Such differences between analytes are well understood by those skilled in the art and are taken into account when determining a predetermined minimum concentration for a particular assay.

In general for the B vitamins, the predetermined minimum concentration will be at least 0.1 ng/ml and is determined for a particular desired analyte. For example, in the case of the assay conditions specifically exemplified herein (see Examples I and II), the predetermined minimum concentration for the assay conditions employed is 0.16 ng/ml for vitamin B₁; 0.75 ng/ml for vitamin B₂; 1.05 ng/ml for vitamin B₃; 1.29 ng/ml for vitamin B₅; 0.33 ng/ml for vitamin B₆; and 0.67 ng/ml for PABA. The upper range for an analyte will generally be in the range of 2000 ng/ml. However, as discussed above, one skilled in the art can readily determine an appropriate concentration of extracted analyte that falls within a quantifiable range such as the linear range using routine methods. A predetermined minimum concentration for a B vitamin is generally at least any of 0.1 ng/ml, 0.2 ng/ml, 0.3 ng/ml, 0.4 ng/ml, 0.5 ng/ml, 0.6 ng/ml, 0.7 ng/ml, 0.8 ng/ml, 0.9 ng/ml, 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1000 ng/ml, 1500 ng/ml, 2000 ng/ml, or even higher, so long as the concentration falls within the quantifiable range of the assays of the method. For those test samples that are solids, it is understood that the solids can be dissolved and/or extracted into a volume suitable to obtain a desired concentration, as discussed above, including dilution to an appropriate concentration after initially dissolving and/or extracting the solid. Similarly, it is understood that a test sample that is a mixture of solids and liquids, or any type of consistency including emulsions, suspensions, and the like, can be dissolved and/or extracted to achieve a desired concentration.

The vitamin standard such as a B vitamin standard contains known, predetermined quantities of desired analytes such as one or more of the various B vitamins. By utilizing isotopically labeled standards, high sensitivity MS analysis can be used to quantify the level of vitamins in a test sample by comparison to the known amount of standard added to the sample. The amount of isotopically labeled standard to be added, as with the predetermined minimum concentration, is based on the limit of quantitation (LOQ) as well as the linear range of detection for the assay conditions being employed for mass spectrometry (MS), as discussed above. Due to the costs of the isotopically labeled standards, it is generally desired to add a sufficient amount of the standard to be above the LOQ and within the linear range of detection while not adding excessive amounts so as not to waste isotopically labeled reagent and increase costs. As discussed above with respect to a predetermined minimum concentration, one skilled in the art can readily determine an appropriate amount of isotopically labeled standard to add to a test sample sufficient to detect the standard and determine the level of analytes in the test sample by comparison to the standard. By comparing the ratio of test analyte to the known quantity of vitamin standard added to the test sample, the level of test sample analyte can readily be determined using MS analysis by comparing the amount to the isotopically labeled standard. Methods of using MS for quantifying the amount of analyte in a test sample by comparison to an isotopically labeled form of the corresponding molecule are well known to those skilled in the art, as disclosed herein.

Such known quantities are generally in the range of 5000 ng/ml to 1 ng/ml, and can contain, for example, a vitamin standard comprising 5000 ng/ml or less of each of the vitamins in the vitamin standard, for example, 4000 ng/ml or less 3000 ng/ml or less, 2000 ng/ml or less, 1000 ng/ml or less, 500 ng/ml, 400 ng/ml or less, 300 ng/ml or less, 200 ng/ml or less, 100 ng/ml or less, 50 ng/ml or less of each, 25 ng/ml or less, 20 ng/ml or less, 15 ng/ml or less, 10 ng/ml or less, 5 ng/ml or less, or 1 ng/ml or less of the vitamins in the vitamin standard. Generally the linear range for the analysis methods disclosed herein are from 5000 ng/ml to 10 ng/ml and therefore represent a desirable range for the methods of analysis disclosed herein, in particular a range of 500 ng/ml to 10 ng/ml. It is understood that each of the vitamins in the standard can be added in the same amount or can be added in different amounts for each of the vitamins in the standard so long as the amount added is suitable for the methods of analysis. For example, 50 ng of one vitamin can be added and 45 ng of another vitamin, or 100 ng of another vitamin, and so forth, so long as the amount added is known and suitable for quantification of the corresponding analytes in a test sample. Methods of determining a suitable amount of a molecule to include in a standard are well known to those skilled in the art, depending on the methods of analysis used, the sensitivity and limit of detection of the instruments used, and the like. Such suitable amounts can be determined using methods well known to those skilled in the art, for example, using standard curves to determine the amounts suitable to generate reproducible and quantitative analysis of analytes, as disclosed herein (see Examples).

Although a vitamin standard can include a single standard, generally it is desirable to include more than one type of vitamin in the vitamin standard to allow simultaneous or parallel analysis of the same sample for the presence and quantity of more than one vitamin. Thus, a vitamin standard generally contains one or more vitamins and can contain a plurality of vitamins. In a particularly useful embodiment, the vitamin standard can contain each of vitamin B₁, vitamin B₂, vitamin B₃, vitamin B₅, vitamin B₆ and p-aminobenzoic acid (PABA).

By utilizing isotope labels and mass spectrometry, the methods of the invention allow efficient and sensitive analysis of complex samples such as foods or drink products for the presence of micronutrients or vitamins such as B vitamins. In contrast to analysis of pharmaceuticals, where relatively homogeneous samples are generally tested, food samples are often complex and non-homogeneous. For example, food samples often contain solids or are suspensions or colloids. Due to the general lack of homogeneity in complex samples such as foods, a larger test sample that accounts for the lack of homogeneity is often required in order to accurately determine the level of analyte representative of the entire sample. Traditionally, sample analysis for complex samples such as foods has generally required the addition of an internal standard into the test sample prior to processing to account for possible analyte loss during processing of the test sample for analysis. As disclosed herein, by efficiently extracting micronutrients such as B vitamins from a sample into a fraction representative of the whole starting sample suitable for subsequent analysis by mass spectrometry, the methods of the invention advantageously allow the use of only a portion of the sample and a significantly smaller amount of isotope labeled internal standard, which is added subsequent to sample processing, thereby reducing costs for MS analysis while maintaining precision of the assay. Thus, adding the isotope label after initial extraction rather than to the starting test sample allows for cost effective analysis by not wasting costly isotope standard while still allowing for a large sample size to be analyzed, thereby taking into account sample heterogeneity. It is understood that one skilled in the art can readily determine a sufficient sample size that accounts for sample heterogeneity and is representative of the amount of analytes in the test sample suitable for extraction and quantitative assays using methods of the invention. Generally, a portion is a small fraction or volume of the total sample and is all that is required when analytes have been efficiently extracted by the methods disclosed herein, for example, a volume of 10 ml or less, 5 ml or less, 4 ml or less, 3 ml or less, 2 ml or less, in particular 1 ml or less, 0.9 ml or less, 0.8 ml or less, 0.7 ml or less, 0.75 ml or less, 0.6 ml or less, 0.5 ml or less, 0.45 ml or less, 0.4 ml or less, 0.35 ml or less, 0.3 ml or less, 0.25 ml or less, 0.2 ml, 0.15 ml or less, or 0.1 ml less, so long as a sufficient volume suitable for subsequent and accurate analysis of analytes is employed. Thus, depending on the volume of the extracted sample, the portion of the sample used for analysis, in which the internal standard is added, can be 1/10, 1/20/ 1/50, 1/100, 1/200, 1/300, 1/400, 1/500, 1/1000, 1/2000, 1/5000, or even 1/10,000 volume of the sample, as appropriate for subsequent analysis. It is understood that various volumes or proportions of the total sample can be used, as desired, so long as the amount is suitable for analysis using the methods of the invention, as disclosed herein.

In the methods of the invention, the vitamin fraction can be obtained by extraction with an acidic aqueous solution. In general, a sample is treated to extract one or more vitamins or micronutrients from a sample into a soluble fraction, which is particularly useful for a sample containing solid materials, and can also be used to remove materials that could precipitate in a buffer used in a subsequent analysis step, for example, to remove proteins that could precipitate during subsequent analysis. As disclosed herein, the inclusion of acid in the aqueous extraction solution of B vitamins stabilizes the extracted vitamins and facilitates analysis of the aqueous extract. The addition of acid to the test sample or inclusion of acid in the vitamin extraction solvent provides analyte stability, thereby allowing the extracted vitamin fraction to accurately reflect the analyte concentration in the starting sample and therefore allowing the use of only a portion of the sample, with addition of an isotope standard to a small volume of extract rather than a large volume of starting sample.

If desired, the acidic aqueous solution can be mixed with an organic solvent such as methanol or acetonitrile, or other suitable organic solvents. In a particular embodiment, the vitamin fraction is obtained by extraction with an acidic alcohol solution, such as an acidic methanol solution or other suitable alcohol, or acidic aqueous solution to which an organic solvent such as acetonitrile is added. Suitable acids include, but are not limited to, HCl, organic acids such as acetic acid, and the like. Exemplary solvents include, but are not limited to, an acidic aqueous solution, for example, 3 to 10 mM acid, for example, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, and the like. For example, a solvent can contain 3 to 10 mM HCl and 0 to 20% methanol, for example, a 5 mM HCl/20% methanol solution can be used to obtain a vitamin fraction, as disclosed herein. Suitable alcohol concentrations include, but are not limited to, 1%, 2%, 5%, 10% 15%, 20% 25%, and the like, including exemplary alcohols such as methanol, ethanol, butanol, and the like. It is well understood to those skilled in the art that various acids in a suitable concentration range, including but not limited to those disclosed herein, including combinations of acids, can be used to extract a vitamin fraction such as a vitamin B fraction, and such extraction buffers can also include one or more organic solvents such as those disclosed herein or well known to those skilled in the art that are suitable for extraction of a vitamin fraction.

The methods of the invention are useful for analyzing micronutrients such as vitamins in complex samples such as foods, including supplemented foods in which a food product is supplemented with desired additives such as micronutrients, dietary supplements, or any type of consumable product containing an analyte of interest. Thus, the methods of the invention are useful for analyzing consumable products, whether for human or animal consumption, such as food samples, drink samples, dietary supplement samples, and the like. For example, the methods can be used to analyze drinks such as milk or milk-derived products. As used herein, milk or a milk derived product includes whole milk or processed forms of milk such as those with reduced fat levels or skim milk, or condensed or powdered milk, and the like. One skilled in the art readily understands the meaning of a milk or milk-derived product. The methods of the invention are also particularly useful for analysis of infant formula. Other samples, include, but are not limited to, fortified drinks, including sports drinks, cereals, or any consumable product such as a food product or drink product in which it is desired to measure the content of one or micronutrients such as vitamins.

In an additional embodiment, the invention provides a method for determining the level of B vitamins in a test sample. Such a method can include the steps of obtaining a B vitamin fraction of a test sample by extraction with an acidic alcohol solution; adding a B vitamin standard to a portion of the B vitamin fraction, the B vitamin standard comprising one or more B vitamins and can contain a plurality of B vitamins selected from vitamin B₁, vitamin B₂, vitamin B₃, vitamin B₅, vitamin B₆ and p-aminobenzoic acid (PABA), wherein the B vitamin standard comprises 500 ng/ml or less of each of the B vitamins in the B vitamin standard and wherein the B vitamins in the B vitamin standard comprise an isotope label comprising 50 ng/ml or less of each of the isotopically labeled B vitamin standards; and determining the level of B vitamins in the test sample corresponding to one or more of the B vitamins in the B vitamin standard using mass spectrometry by comparison of the amount of one or more B vitamins in the test sample to the B vitamin standard.

It is understood that modifications which do not substantially affect the activity of the various embodiments of this invention are also provided within the definition of the invention provided herein. Accordingly, the following examples are intended to illustrate but not limit the present invention.

### EXAMPLE I

### Assay Method for B Vitamins

This example describes an exemplary assay method for measurement of B vitamins.

The overall principle of the assay is that B vitamins (B₁, B₂, B₃, B₅, B₆ and PABA) are extracted by acidic solvent (5 mM HCl/20% methanol). The extract is adjusted to the pH range of 4.5 to 5.5 for protein precipitation. No further purification is required except filtering with a 0.45 μm membrane filter. Isotope internal standard of B₁, B₂, B₃, B₅, B₆ and PABA are mixed with the filtered solution for LC/MS/MS measurement.

Each run generally contains a minimum of 10% quality assurance samples, which can include duplicate analysis or validated control samples. The same isotope internal standard solution of 1 μg/mL is used for samples and standard solutions. The isotope internal standards at a concentration of 50 ng/mL are included in the final sample solutions, as well as the standard solutions used for calibration.

A chromatography column such as a Zorbax extended C18, 3.5 μm, 2.1×100 mm (Agilent; Santa Clara Calif.) or similar column is used. An HPLC System such as the Shimadzu HPLC system (LC-20AD) (Shimadzu; Kyoto Japan) or similar system is used. Mass spectrometry is carried out on a 4000-Q TRAP LC/MS/MS System (Sciex; Concord Ontario) triple quadrupole tandem mass spectrometer or similar instrument.

The following reference standards are used: thiamine hydrochloride; riboflavin; niacin; calcium pantothenate; pyridoxine hydrochloride; p-Aminobenzoic acid (PABA); nicotinamid; pyridoxal hydrochloride; pyridoxamine dihydrochloride; 4-pyridoxic acid (Sigma-Aldrich; St. Louis Mo.); thiamine chloride (4,5,4 methyl-¹³C, 99%) (Cambridge Isotope Laboratories, Inc.; Andover Mass.); nicotinamide-2,4,5,6,-d4, 98% atom D (C/D/N Isotopes Inc.; Pointe-Claire, Quebec, Canada); nicotinic-d4 acid, 98% atom D (Sigma-Aldrich); pantothenic acid, calcium salt monohydrate (Beta-alanyl-¹³C3, 99%; ¹⁵N, 98%) (Cambridge Isotope Laboratories); pyridoxine-5′,5′-d2 HCl, 98% atom D (C/D/N Isotopes); 4-aminobenzoic-2,3,5,6,-d4 acid, 98% atom D (CDN Isotopes).

Stock and working standard solutions were prepared as follows.

Solvent Preparation: Preparation of 1M HCl: A volume of 8.33 mL of concentrated HCl is added to a 100-mL volumetric flask and brought to 100 mL volume with purified water. The solution is stable for 3 months when stored at ambient temperature. Preparation of 50 mM HCl: A volume of 5.0 mL 1M HCl is added to a 100-mL volumetric flask and brought to 100 mL volume with purified water. The solution is stable for 3 months when stored at ambient temperature. Preparation of Extraction Solvent, 5 mM HCl/20% methanol: A volume of 400 mL methanol is added to a 2000-mL volume flask. A volume of 10 mL of 1 M HCl solution is added and brought to 2000 mL volume with purified water. The solution is stable for 1 week when stored at ambient temperature.

Stock Standard Solutions: Thiamine Hydrochloride Stock Standard, 1000 μg/mL: 101.0 mg of reference standard is weighed into a 100-mL volumetric flask, dissolved in 50-mM HCl and diluted to volume with the same solvent. The stock standard is stable for 3 months if stored in a refrigerator set to maintain 2-8° C. Riboflavin Stock Standard, 100 μg/mL: 25.0 mg of reference standard is weighed into a 250-mL volumetric flask, dissolved in 50 mM HCl and diluted to 250 mL volume with the same solvent. The stock standard is stable for 3 months if stored in a refrigerator set to maintain 2-8° C. Niacin Stock Standard, 1000 μg/mL: 100.2 mg of reference standard is weighed into a 100-mL volumetric flask, dissolved in 50 mM HCl and diluted to 100 mL volume with the same solvent. The stock standard is stable for 3 months if stored in a refrigerator set to maintain 2-8° C. Nicotinamide Stock Standard, 1000 μg/mL: 100.7 mg of reference standard is weighed into a 100-mL volumetric flask, dissolved in 50 mM HCl and diluted to 100 mL volume with the same solvent. The stock standard is stable for 3 months if stored in a refrigerator set to maintain 2-8° C. Calcium Pantathenic Acid Stock Standard, 1000 μg/mL; 100 mg of reference standard is weighed into 100-mL volumetric flask, dissolved in 50 mM HCl and diluted to 100 mL volume with the same solvent. The stock standard is stable for 3 months if stored in a refrigerator set to maintain 2-8° C. Pyridoxine Stock Standard, 1000 μg/mL: 100 mg of reference standard is weighed into a 100-mL volumetric flask, dissolved in 50 mM HCl and diluted to 100 mL volume with the same solvent. The stock standard is stable for 3 months if stored in a refrigerator set to maintain 2-8° C. Pyridoxal Hydrochloride Stock Standard, 1000 μg/mL: 100 mg of reference standard is weighed into 100-mL volumetric flask; dissolved in 50 mM HCl and diluted to 100 mL volume with the same solvent. The stock standard is stable for 3 months if stored in a refrigerator set to maintain 2-8° C. Pyridoxamine Dihydrochloride Stock Standard, 1000 μg/mL: 101.0 mg of reference standard is weighed into a 100-mL volumetric flask, dissolved in 50 mM HCl and diluted to 100 mL volume with the same solvent. The stock standard is stable for 3 months if stored in a refrigerator set to maintain 2-8° C. 4-pyridoxic Acid Stock Standard, 1000 μg/mL: 101.0 mg of reference standard is weighed into a 100-mL volumetric flask, dissolved in 50 mM HCl and diluted to 100 mL volume with the same solvent. The stock standard is stable for 3 months if stored in a refrigerator set to maintain 2-8° C. PABA Stock Standard, 1000 μg/mL: 101.0 mg of reference standard is weighed into a 100-mL volumetric flask, dissolved in 50 mM HCl and diluted to 100 mL volume with the same solvent. The stock standard is stable for 3 months if stored in a refrigerator set to maintain 2-8° C.

Intermediate Standard Solutions. Intermediate Standard Solution A: 18 μg/mL for Nicotinamide and B₅, and 4.5 μg/mL for B₁, B₂, Niacin, Pyridoxine, Pyridoxal, Pyridoxamine, Pyridoxic Acid and PABA. Into a 100-mL volume flask is added 1.8 mL of the stock solutions of nicotinamide and B₅, 4.5 mL of the stock solution of B₂, 0.45 mL of the stock solutions of B₁, niacin, pyridoxine, pyridoxal, pyridoxamine, pyridoxic acid and PABA. Dilute to volume with 50 mM HCl. This standard solution is stable for 2 months when stored in a refrigerator set to maintain 2-8° C. Intermediate Standard Solution B: 3 μg/mL for Nicotinamide and B₅, 0.75 μg/mL for B₁, B₂, Niacin, Pyridoxine, Pyridoxal, Pyridoxamine, Pyridoxic Acid and PABA. A volume 0.833 mL of the Intermediate Stock Solution A is added into a 5-mL volume flask, and 1 mL of 50 mM HCl is added. Dilute to volume with purified water. This standard solution is stable for 1 week when stored in a refrigerator set to maintain 2-8° C.

Stock Solutions of Isotope Internal Standards. Thiamine Chloride (4,5,4 metyl-¹³C) Stock Standard, 1000 μg/mL: Approximately 5 mg of reference standard is weighed into a 15-mL glass vial and dissolved in 5 mL of 50 mM HCl. The stock standard is stable for 2 years when stored in a refrigerator set to maintain 2-8° C. Nicotinamide-2,4,5,6,-d4 Stock Standard, 1000 μg/mL: Approximately 5 mg of reference standard is weighed into a 15-mL glass vial and dissolved in 5 mL of 50-mM HCl. The stock standard is stable for 2 years when stored in a refrigerator set to maintain 2-8° C. Pantothenic Acid, Calcium Salt Monohydrate, 1000 μg/mL: Approximately 5 mg of reference standard is weighed into a 15-mL glass vial and dissolved in 5 mL of 50 mM HCl. The stock standard is stable for 2 years when stored in a refrigerator set to maintain 2-8° C. Pyridoxine-5′,5′-d2 HCl, Stock Standard, 1000 μg/mL: Approximately 5 mg of reference standard is weighed into a 15-mL glass vial and dissolved in 5 mL of 50-mM HCl. The stock standard is stable for 2 years when stored in a refrigerator set to maintain 2-8° C. 4-Aminobenzoic-2,3,5,6,-d4 acid (PABA), Stock Standard, 1000 μg/mL: Approximately 5 mg of reference standard is weighed into a 15-mL glass vial and dissolved in 5 mL of 50-mM HCl. The stock standard is stable for 2 years when stored in a refrigerator set to maintain 2-8° C.

Mixed Internal Standard Solutions. Mixed Internal Standard Solution A: 10 μg/mL for Isotope Internal Standard of B₁, Nicotinamide, Niacin, B₅, B₆ and PABA. A volume of 0.100 mL of the stock isotope internal standard solutions is placed into a 10-mL glass vial and diluted to volume with 50 mM HCl. This mixed isotope standard solution is stable for 3 months if stored in a refrigerator set to maintain 2-8° C. Mixed Internal Standard Solution B: 1 μg/mL for Isotope Internal Standard of B₁, Nicotinamide, Niacin, B₅, B₆ and PABA. 1.0 mL of the 10 μg/mL mixed isotope internal standard solution is placed into a 10-mL volume flask and 1.0 mL of 50 mM HCl is added. Dilute to volume with purified water. This mixed isotope standard solution is stable for one month if stored in a refrigerator set to maintain 2-8° C. The same internal standard solution should be used for samples as well as standard solutions in the same batch.

Working Solutions. Preparation of Dilution Solution A for Standard Solution: 5 mM HCl/5% Methanol/50 ng/mL isotope standards. A volume of 1.0 mL of 50 mM HCl is placed into a 10 mL volume flask, and 0.5 mL of the 1 μg/mL isotope standard solution and 0.5 mL of methanol (MeOH) are added. The volume is brought to 10 mL with purified water. This fresh solution should be prepared daily. Preparation of Dilution Solution B for Sample Solution: 6.7 mM HCl/66.7 ng/mL isotope standards. A volume of 1.667 mL of the 1 μg/mL isotope standard solution is placed in a 25-mL volume flask, and 3.35 mL of 50 mM HCl is added. The volume is brought to 25 mL with purified water. The solution contains 66.7 ng/mL isotope standards in 6.7 mM HCl. This fresh solution should be prepared daily.

Preparation of Calibration Solutions. Preparation of Calibration Solution STD1: 1.0 mL of the Intermediate Standard Solution B is placed in a 5-mL volume flask, a volume of 0.25 mL of the 1 μg/mL isotope standard solution is added, and 0.25 mL of MeOH is added. Dilute to volume with purified water. About 1 mL is transferred to a 1.5-mL HPLC sample vial. This fresh solution should be prepared daily. Preparation of Calibration Solution STD2: A volume of 0.667 mL of STD1 is placed into a 1.5-mL HPLC sample vial, and 0.333 mL of the Dilution Solution A is added. This fresh solution should be prepared daily. Preparation of Calibration Solution STD3: A volume of 0.333 mL of STD1 is placed into a 1.5-mL HPLC sample vial, and 0.667 mL of the Dilution Solution A is added. This fresh solution should be prepared daily. Preparation of Calibration Solution STD4: A volume of 0.417 mL of STD1 is placed into a 15-mL glass vial, and 4.583 mL of the Dilution Solution A is added. About 1 mL is transferred to a 1.5-mL HPLC sample vial. This fresh solution should be prepared daily. Preparation of Calibration Solution STD5: A volume of 0.100 mL of STD4 is placed into a 1.5-mL HPLC sample vial, and 0.900 mL of the Dilution Solution A is added. The standard solutions contain 50.0 ng/mL isotope standards in 5.0 mM HCl. This fresh solution should be prepared daily.

Sample Preparation. Sample Extraction: (1) Weigh 1 gram milk powder and put it in a 125 mL flask. (2) Add 50 mL of 5 mM HCl/20% methanol extraction solvent. (3) Vortex and sonicate for 30 minutes. (4) Check pH value with a pH meter and adjust to 4.5 to 5.5 by using 1 N HCl or 1N NaOH, generally a couple of drops, if necessary. (5) Filter and collect 1 mL of the extraction solution with a 0.45 um polytetrafluoroethylene (PTFE) membrane. (6) Take 0.25 mL of the filtered solution to a 1.5-mL HPLC sample vial, followed by adding 0.75 mL of Dilution Solution B. The sample solutions contain 50.0 ng/mL isotope standards in 5.0 mM HCl. Liquid milk products and infant formulas are similarly processed.

Instrument Parameters. The following parameters are starting points used to obtain desirable chromatographic results. Adjustments can be made, as necessary, to improve chromatographic or MS results.

HPLC Parameters: Column, Zorbax Extended C18, 3.5 μm, 2.1×100 Mm (Agilent); Mobile Phase A, 0.1% acetic acid in water; Mobile Phase B: 0.1% acetic acid in methanol; Flow Rate: 0.2 ml/minute; Injection Volume: 5 μL. The HPLC run is started with phase B at 7% and held until 2.9 min, followed by a linear gradient increasing to 60% at 3.5 min. The 60% phase B is held until 7.0 min before decreasing to 7%, allowing equilibrium until 9.8 min for the next injection. By this gradient procedure, the analytes can be injected for simultaneous measurement in less than 10 minutes.

MS/MS Parameters: Source parameters: Curtain Gas 40; Voltage 5000; Source Gas 1 & 2: 65; Temperature 500° C. Equilibrate the system and check it by injecting standards until reproducible retention times are obtained. Inject one of the working standards between a maximum of every eight samples.

To calculate the sample concentration, use the formula C×50×(4/1000)=μg/g, where C=Concentration from regression analysis (ng/mL)

### EXAMPLE II

### Measurement of Water Soluble B Vitamins in Infant Formula by Liquid Chromatography Tandem Mass Spectrometry (LC/MS/MS)

This example describes the measurement of water soluble B vitamins in a complex food sample.

Briefly, B vitamins, B₁, B₂, B₃, B₅, B₆ and PABA, are extracted by acidic solvent. The extract is adjusted to the pH range of 4.5 to 5.5 for protein precipitation. No further purification is required except filtering with a 0.45 μm membrane filter. An isotope internal standard is mixed with the filtrate for LC/MS/MS analysis.

Generally in LC/MS analysis, the isotope internal standards (IS) are added before extraction to account for extraction efficiency. The isotope standards are generally expensive due to the cost of synthesis. In food analysis, large sample sizes are generally needed, for example, 1-5 grams, as compared to drug analysis, which are usually more homogenous samples. In the analysis described below, the internal standards are added after the extraction procedure. If the traditional manner of adding internal standard was used in this case, the amount of IS could be 50 μg (5 gram size/1000 mL (5 mg/ml) in extraction solvent) for each IS vitamin to achieve a final internal standard concentration of 50 ng/ml (50,000 ng in 1000 ml=50 μg). However, since only 1 mL or less is needed for the LC/MS/MS measurement, more than 99.5% of the internal standards are wasted using the traditional method. By stabilizing the analytes in extraction solution, the internal standard is only added to a small fraction of the total sample such as a 1.0 mL fraction of the total sample volume, thereby significantly reducing the cost of analysis.

In more detail, the B vitamins B₁, B₂, B₃, B₅, B₆ and PABA in infant formula were measured by LC/MS/MS simultaneously with a single injection. The instrument used was an atmospheric pressure ionization (API)-4000 triple quadrupole tandem mass spectrometer (MDS Sciex; Concord Ontario) with a Shimadzu HPLC system (LC-20AD) (Shimadzu; Kyoto Japan).

For sample preparation, 1 gram milk powder was weighed out and put in a 125 mL flask. A volume of 50 mL of 5 mM HCl/20% methanol extraction solvent was added, and the sample was vortexed and sonicated for 30 minutes. The pH was checked and adjusted to a range of 4.5 to 5.5, if need. It was found that the addition of acid stabilized the extracted B vitamins in that, as disclosed below in more detail, the processed sample was representative of the amount of B vitamins in the starting sample. The extract was filtered with a 0.45 μm polytetrafluoroethylene (PTFE) membrane before measurement.

For addition of internal standard, a volume of 0.25 mL of the filtered solution was transferred to a 1.5 mL HPLC sample vial, and 0.75 mL of solvent containing 66.7 ng/mL of appropriate isotope internal standards were added. The final sample solution contained 50 ng/mL isotope internal standards for each of the B vitamins to be measured. The same amount of standard was included in reference standard solutions.

HPLC separation was utilized to minimize signal depression and allows measurement of the analytes simultaneously with reasonable running time. An HPLC Zorbax (Agilent; Santa Clara Calif.) extended C18 column (2.1×150 mm, 3.5 μm) was used. A gradient HPLC program was employed for an injection of 5 μL of sample solution. The flow rate was 0.2 mL/min, with mobile phase A of 0.1% acetic acid in water and phase B of 0.1% acetic acid in methanol. The HPLC run was started with phase B at 7% and held until 2.9 min, followed by a linear gradient increasing to 60% at 3.5 min. The 60% phase B was held until 7.0 min before decreasing to 7%, allowing equilibrium until 9.8 min for the next injection. By this gradient procedure, the analytes can be injected for simultaneous measurement in less than 10 minutes.

For mass spectrometry, common parameters were optimized, including electrospray voltage, 5500 V (suitable range 5000-5500 V); temperature, 550° C. (suitable range 500-550° C.); gas 1, 65 psi; gas 2, 60 psi (suitable range 50-65 psi); curtain gas, 25 psi (suitable range 25-40 psi). The parameters of mass analyzer for each analyte are listed in Table 1.

TABLE 1
Parameters for Mass Spectrometry Measurement.

| Analyte | Q1 (amu) | Q3 (amu) | DP | EP | CE | CXP |
|---|---|---|---|---|---|---|
| B₁ | 265.20 | 122.10 | 35.0 | 10.0 | 21.0 | 22.0 |
| B₂ | 377.20 | 243.20 | 70.0 | 10.0 | 35.0 | 15.0 |
| B₃ (acid) | 123.10 | 80.00 | 60.0 | 10.0 | 30.0 | 14.0 |
| B₃ (mide) | 124.10 | 80.00 | 60.0 | 10.0 | 30.0 | 14.0 |
| B₅ | 220.20 | 94.00 | 40.0 | 10.0 | 22.0 | 16.0 |
| B₆ | 170.20 | 134.10 | 45.0 | 9.00 | 31.0 | 7.00 |
| PABA | 138.10 | 94.00 | 45.0 | 9.00 | 20.0 | 9.00 |
| Pyridoxamine | 169.10 | 134.10 | 38.0 | 4.00 | 18.0 | 9.00 |
| Pyridoxal | 168.10 | 150.10 | 35.0 | 4.00 | 16.5 | 8.80 |
| Pyridoxic | 184.10 | 148.00 | 43.0 | 4.00 | 28.0 | 8.21 |
| acid | | | | | | |

Q1, quadrupole 1
Q3, quadrupole 3
DP, deflector potential
EP, entrance potential
CE, collision energy
CXP, collision cell exit potential

Additional optimization was performed, and the results are shown in Table 2.

TABLE 2
Optimized Parameters for MS Analysis Under Positive Ion Mode.

| | Precursor | | | | | |
|---|---|---|---|---|---|---|
| | Ion | Product Ion | DP | EP | CE | CXP |
| B₁ | 265.2 | 122.1, 144.2, 81.2 | 40 | 9 | 21 | 6 |
| B₂ | 377.2 | 243.2, 359.2, 99.1 | 70 | 11 | 34 | 15 |
| B₃(acid) | 124.1 | 80.2, 78.2, 106.1 | 60 | 9 | 30 | 14 |
| B₃ | 123.1 | 80.1, 78.0, 96.0 | 55 | 9 | 30 | 7 |
| B₅ | 220.2 | 90.1, 202.2, 184.2 | 44 | 9 | 22 | 16 |
| B₆ | 170.2 | 134.1, 152.2, 124.2 | 45 | 10 | 29 | 7 |
| PABA | 138.1 | 120.1, 94.1, 81.2 | 45 | 10 | 20 | 9 |

Source parameters: Curtain gas 40; voltage 5000; source gas 1 and 2, 50; temperature 500° C.
The first product ions shown in bold are the ions used for quantitative measurement of the analytes.

Eight concentration levels were used for the calibration curve during method development. The coefficient was typically 0.9999 and showed good linearity for the analytes. For routine analysis, generally 5 standard solutions are used for the calibration.

For method validation, infant formula reference material SRM (standard reference material) 1846 (NIST) was used for the validation. The results for method validation showed good agreement. Spike recovery was investigated and typically found to be around 90% to 110%.

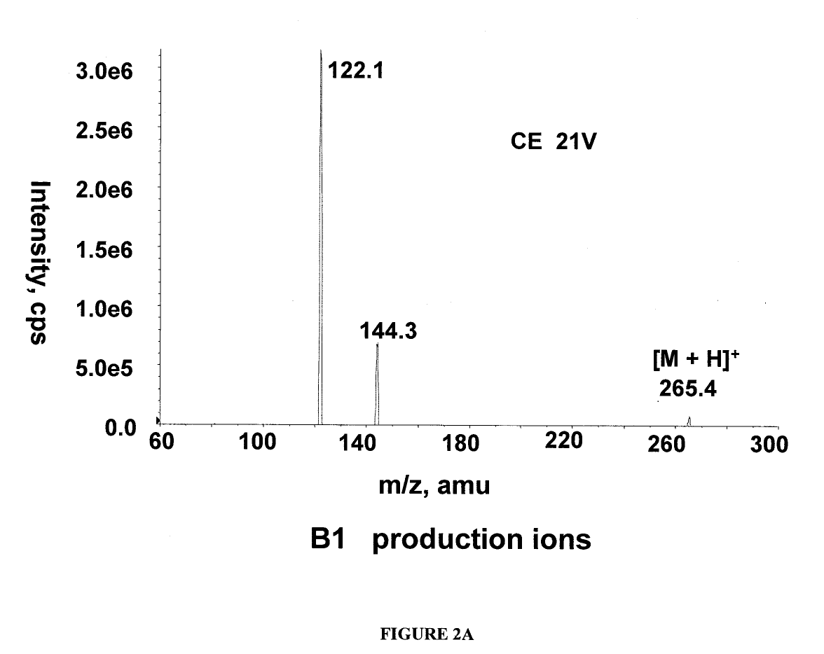

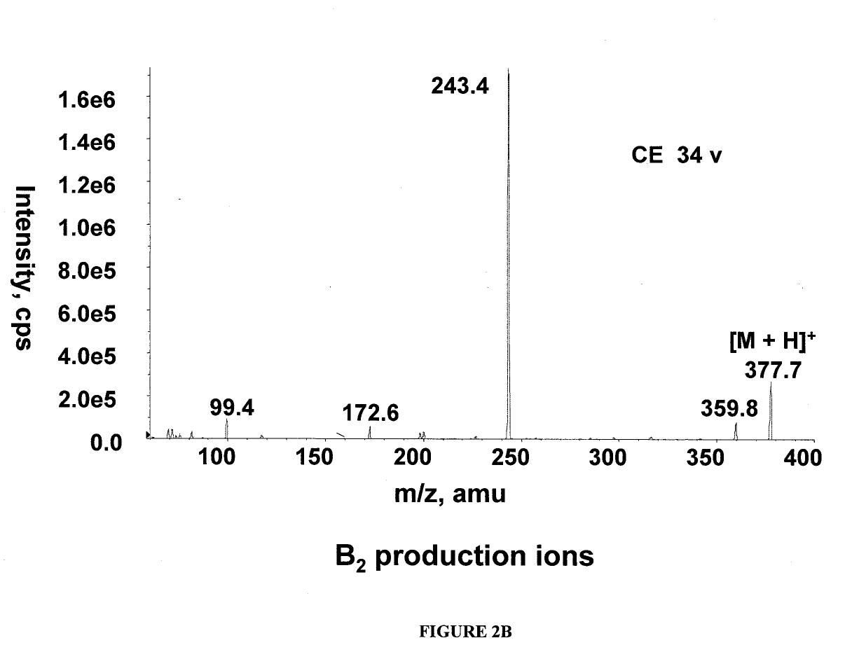

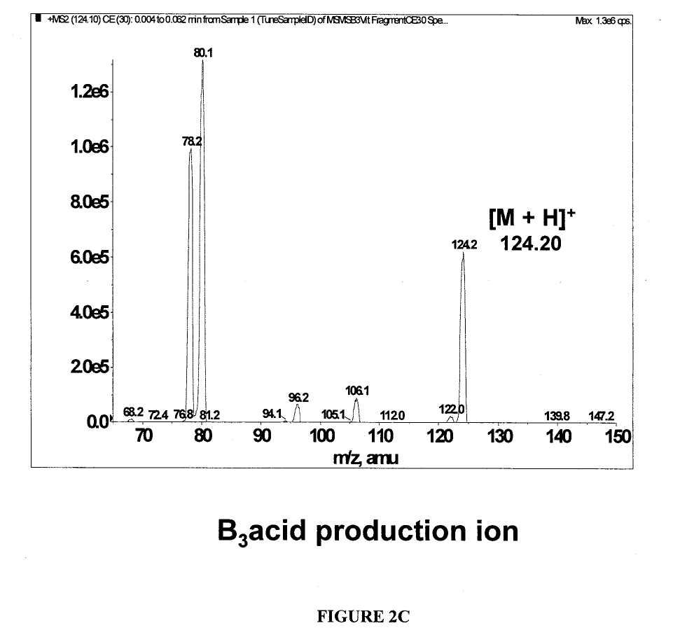

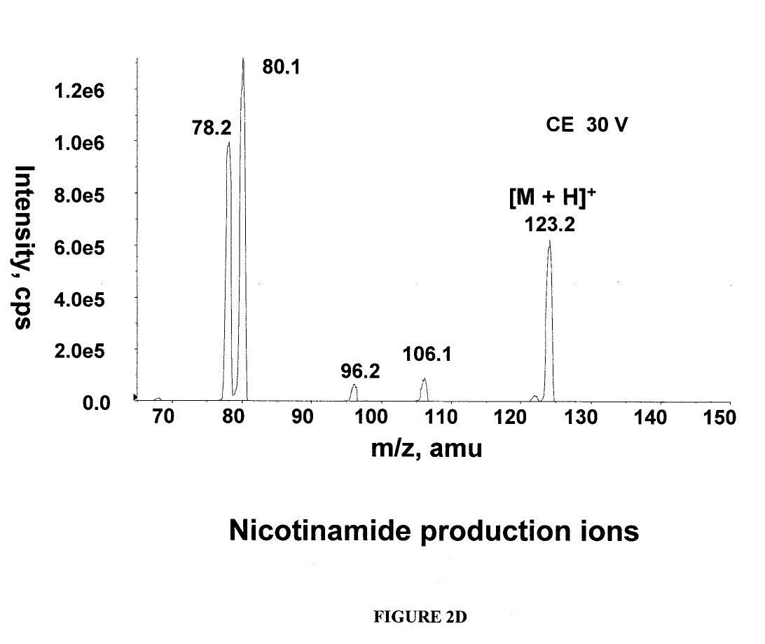

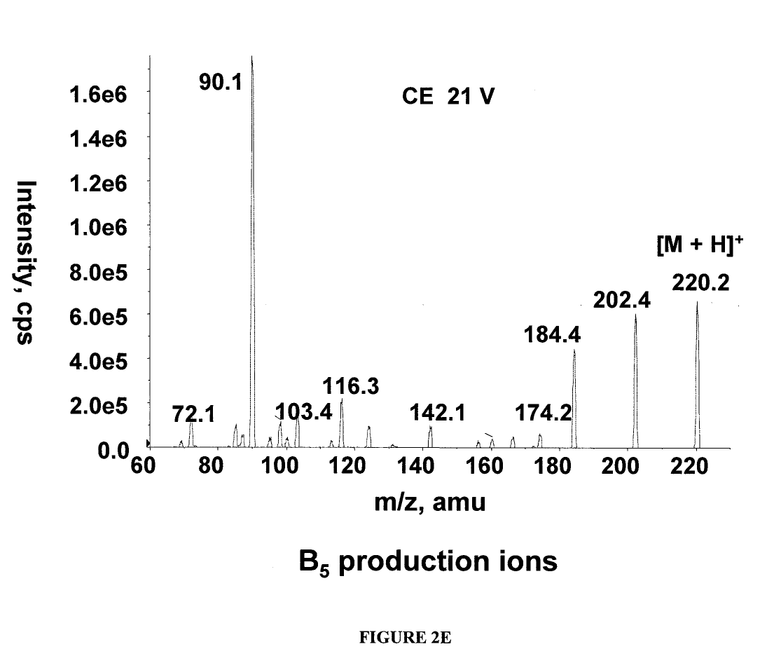

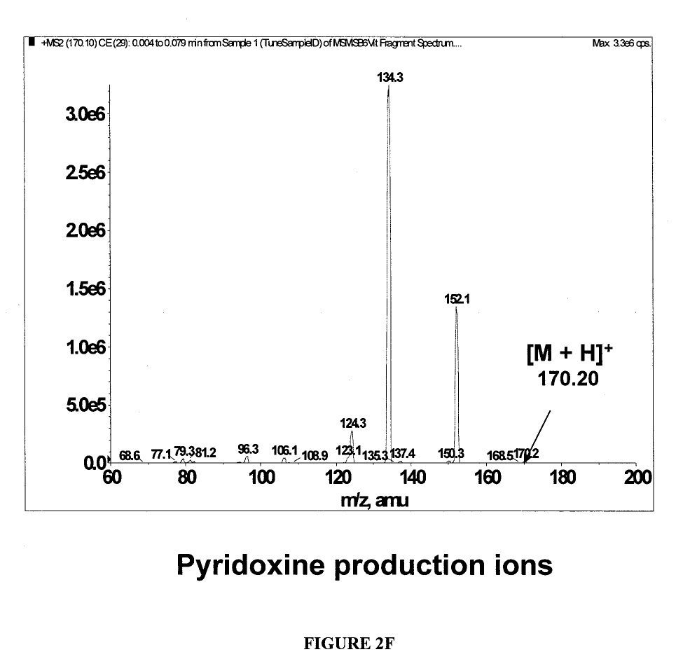

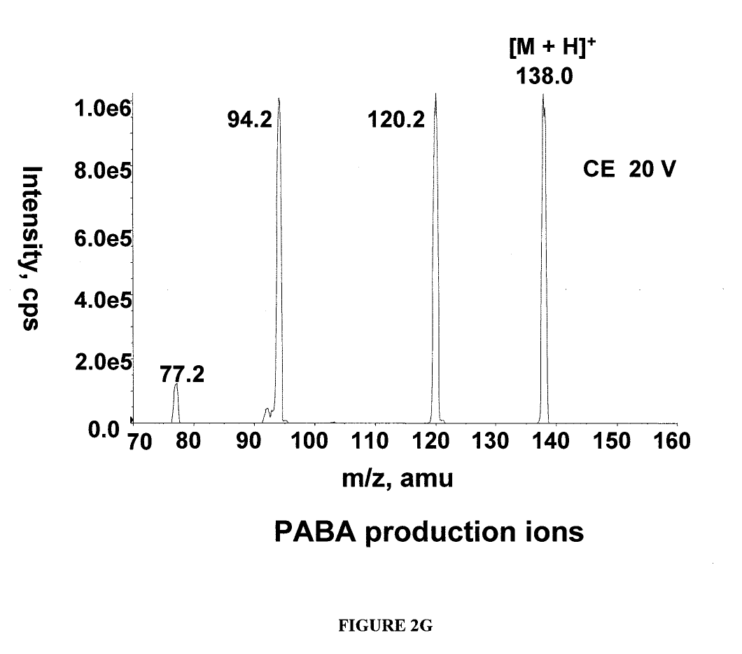

Multiple reaction monitoring (MRM) for the analytes. Measurements were made under positive ion mode. The fragmentation ions of these analytes are shown in FIG. 2. The most abundant reactions used for quantification are given in Table 1.

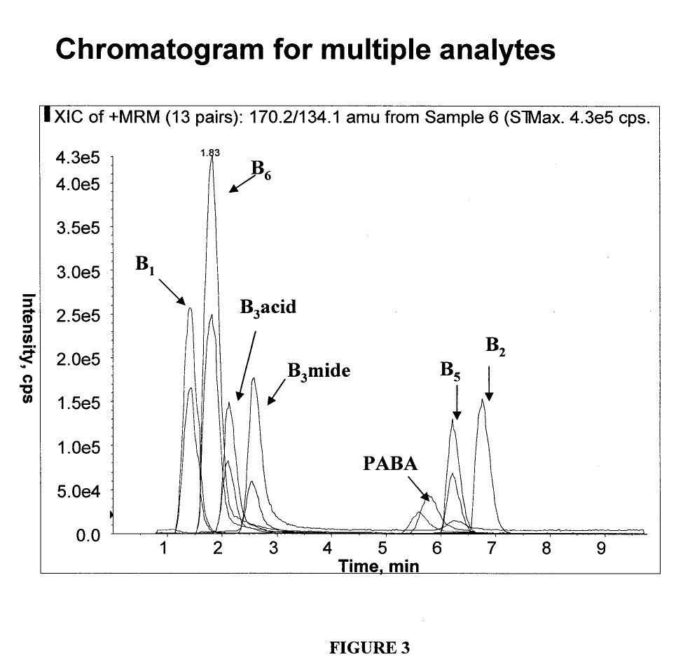

FIG. 3 shows a representative chromatogram for the analytes. Under the optimized HPLC conditions, all of the analytes can be measured in less than 10 minutes, including about 3 minutes for equilibrium between analytical runs.

Accuracy and precision. The accuracy of the method was validated by analyzing SRM 1846 infant formula reference material from the NIST. The results are comparable with the recommended values obtained by microbiological method (Table 3). Generally, microbiological methods report higher values due to less specificity compared with LC/MS/MS.

TABLE 3
Measurement of B vitamins in SRM 1846 by LC/MS/MS (μg/g).

| Sample | B₁ | B₂ | B₃mide | B₃acid | B₅ | B₆ |
|---|---|---|---|---|---|---|
| SRM1 | 8.92 | 16.94 | 54.60 | 0.55 | 33.00 | 7.30 |
| SRM2 | 8.94 | 15.88 | 53.60 | 0.37 | 40.20 | 6.98 |
| SRM3 | 8.98 | 16.20 | 56.60 | 0.54 | 35.60 | 7.12 |
| SRM4 | 8.90 | 16.06 | 55.60 | 0.37 | 49.40 | 7.50 |
| SRM5 | 9.04 | 16.08 | 54.80 | 0.46 | 40.40 | 7.26 |
| SRM6 | 8.90 | 15.96 | 54.20 | 0.42 | 48.80 | 6.96 |
| SRM7 | 9.18 | 15.92 | 56.60 | 0.48 | 44.20 | 7.46 |
| SRM8 | 9.22 | 16.20 | 55.00 | 0.42 | 42.60 | 7.30 |
| Ave | 9.01 | 16.2 | 55.1 | 0.45 | 41.8 | 7.24 |
| SD | 0.63 | 1.69 | 5.40 | 0.35 | 28.9 | 1.01 |
| RSD, % | 1.40 | 2.10 | 1.96 | 15.3 | 13.8 | 2.78 |
| NIST | 9.72 ± 1.34 | 17.4 ± 1.0 | 63.3 ± 7.6 | NA | 52.9 ± 7.9 | 8.4 ± 1.0 |
| Micro | | 19.3 | 62.8 | | 56 | 10.6 |
| | | 18.8 | 60.0 | | 43.6 | 10.4 |

NIST: certified or reference concentrations
Micro: microbiological results

In addition to testing standard reference materials (SRM), a commercial product of infant formula was also analyzed. The results of the method were compared to those obtained by the microbiological method, as shown in Table 4.

TABLE 4
Results from infant formula sample (8 replicates, μg/g).

| | | | | | | | B₆acid | B₆acid |
|---|---|---|---|---|---|---|---|---|
| | B₁ | B₂ | B₃mide | B₃acid | B₅ | B₆ | 152 | 134 |
| Sam1 | 4.90 | 6.92 | 54.8 | 1.146 | 27.0 | 4.12 | 4.06 | 4.04 |
| Sam2 | 5.02 | 7.00 | 57.2 | 1.162 | 27.6 | 4.20 | 4.22 | 4.22 |
| Sam3 | 5.02 | 6.94 | 57.0 | 1.036 | 27.8 | 4.34 | 4.42 | 4.22 |
| Sam4 | 5.00 | 7.06 | 57.0 | 1.206 | 27.6 | 4.22 | 4.32 | 4.24 |
| Sam5 | 5.18 | 7.20 | 58.6 | 1.126 | 28.4 | 4.34 | 4.50 | 4.34 |
| Sam6 | 4.94 | 7.00 | 56.8 | 1.174 | 27.0 | 4.08 | 4.12 | 4.30 |
| Sam7 | 5.00 | 7.00 | 57.4 | 1.096 | 27.6 | 4.18 | 4.58 | 4.34 |
| Sam8 | 4.94 | 7.08 | 56.8 | 1.768 | 27.4 | 4.24 | 4.28 | 4.34 |
| Ave | 5.00 | 7.03 | 57.0 | 1.21 | 27.6 | 4.22 | 4.31 | 4.26 |
| SD | 0.085 | 0.089 | 1.05 | 0.230 | 0.450 | 0.093 | 0.181 | 0.102 |
| RSD, % | 1.70 | 1.26 | 1.84 | 18.9 | 1.63 | 2.20 | 4.19 | 2.39 |
| Client | 4.1 | 7.2 | 52.0 | | 18.0 | 4.5 | | |
| Micro | | 10.2 | 65.7 | | 27.7 | 7.2 | | |
| HPLC | 4.1-5.0 | | | | | | | |

The accuracy of the method was also evaluated by spiking into a sample, either into SRM 1846 (Table 5) or infant formula (Table 6). The accuracy was also supported by spike recovery that was within the range of 100±6% (see Table 6). The relative standard deviation for 8 measurements was less than 5% typically.

TABLE 5
Spike recovery (%) for vitamins added to SRM 1846.

| | | | | | | | B₆acid | B₆acid | |
|---|---|---|---|---|---|---|---|---|---|
| Sample | B₁ | B₂ | B₃mide | B₃acid | B₅ | B₆ | 166 | 138 | PABA |
| SRMsp1 | 98.5 | 106.9 | 98.1 | 89.0 | 138.3 | 96.0 | 111.1 | 103.5 | 91.1 |
| SRMsp2 | 101.5 | 122.5 | 99.4 | 82.8 | 84.6 | 105.2 | 124.9 | 120.0 | 93.8 |
| SRMsp3 | 109.1 | 121.0 | 100.6 | 90.4 | 169.1 | 106.4 | 118.0 | 110.6 | 93.1 |
| SRMsp4 | 98.5 | 122.7 | 91.4 | 86.8 | 69.1 | 105.7 | 118.5 | 113.3 | 92.1 |
| SRMsp5 | 101.5 | 120.0 | 102.5 | 85.6 | 130.2 | 100.0 | 119.0 | 109.9 | 92.8 |
| SRMsp6 | 109.9 | 124.0 | 99.4 | 81.7 | 93.8 | 104.4 | 115.6 | 115.1 | 93.8 |
| SRMsp7 | 99.8 | 117.0 | 93.8 | 94.8 | 120.4 | 96.3 | 110.4 | 105.9 | 91.6 |
| SRMsp8 | 102.7 | 121.0 | 98.1 | 82.0 | 119.8 | 99.0 | 126.9 | 117.0 | 95.3 |
| Ave | 102.7 | 119.4 | 97.9 | 86.6 | 115.7 | 101.6 | 118.1 | 111.9 | 93.0 |
| SD | 4.46 | 5.45 | 3.63 | 4.59 | 32.14 | 4.29 | 5.86 | 5.56 | 1.37 |

TABLE 6
Spike recovery (%) for the vitamins added to infant formula samples.

| | | | | | | | B₆acid | B₆acid | |
|---|---|---|---|---|---|---|---|---|---|
| Sample | B₁ | B₂ | B₃mide | B₃acid | B₅ | B₆ | 152 | 134 | PABA |
| Spike1 | 102.0 | 95.5 | 98.7 | 102.5 | 98.3 | 99.6 | 97.4 | 104.5 | 84.7 |
| Spike2 | 96.8 | 88.3 | 93.1 | 100.3 | 92.8 | 94.1 | 99.6 | 95.9 | 84.2 |
| Spike3 | 97.3 | 91.3 | 99.7 | 100.9 | 97.7 | 96.1 | 103.8 | 99.6 | 84.0 |
| Spike4 | 107.4 | 94.5 | 100.8 | 102.8 | 96.5 | 96.4 | 100.3 | 99.8 | 85.7 |
| Spike5 | 99.3 | 89.6 | 95.9 | 97.5 | 92.8 | 95.4 | 99.1 | 102.3 | 84.2 |
| Spike6 | 100.5 | 98.7 | 97.3 | 100.0 | 97.1 | 100.3 | 100.1 | 104.8 | 86.4 |
| Spike7 | 91.4 | 87.4 | 91.6 | 93.2 | 84.1 | 83.5 | 96.1 | 97.1 | 82.5 |
| Spike8 | 103.7 | 100.4 | 100.1 | 99.4 | 97.7 | 99.1 | 105.3 | 106.2 | 86.7 |
| Ave | 99.8 | 93.2 | 97.1 | 99.6 | 94.6 | 95.6 | 100.2 | 101.3 | 84.8 |
| SD | 4.86 | 4.85 | 0.03 | 3.07 | 4.77 | 5.33 | 3.04 | 3.78 | 1.40 |

Monitoring different chemical forms. Current microbiological method have less specificity, such as those described in Official Method 961.15 (Modified), Official Methods of Analysis of AOAC International, 17th ed., AOAC International, Gaithersburg Md. (2000). The results using a microbiological method are shown in Table 7. In the case of B6, for example, other similar molecules may have a similar response. It was found that pyridoxamine and pyridoxal had a response as strong as B6 (Table 7). However, these compounds can be distinguished by LC/MS/MS without any problem in real samples such as milk.

TABLE 7
Signal Response in Microbiological Method.

| Compound | Response, % |
|---|---|
| B₆, pyridoxine | 100 |
| Pyridoxic acid | 0 |
| Pyridoxamine | 84 |
| Pyridoxal | 112 |

Limit of Detection. Since pure milk powder containing no vitamins is not available, the detection limit, defined as 3 times of standard deviation (SD), was determined by repeatedly measuring (10 times) a diluted standard solution with a concentration close to blank for the analytes. Lower limit of quantification (LLOQ) was calculated based on 10 times of the standard deviation. Table 8 shows limit of detection (LOD) and LLOQ for B vitamins analyzed.

TABLE 8
Limit of detection (LOD) and lower limit of quantification (LLOQ)
for various B vitamins.

| | B₁ | B₂ | B₃mide | B₃acid | B₅ | B₆ | PABA |
|---|---|---|---|---|---|---|---|
| Average | 0.23 | 0.27 | 0.40 | 0.20 | 2.2 | 0.37 | 024 |
| ng/mL | | | | | | | |
| SD | 0.016 | 0.075 | 0.11 | 0.095 | 0.13 | 0.033 | 0.067 |
| LOD | 0.049 | 0.22 | 0.31 | 0.27 | 0.39 | 0.10 | 0.20 |
| ng/mL | | | | | | | |
| LLOQ | 0.16 | 0.75 | 1.1 | 0.91 | 1.3 | 0.33 | 0.67 |
| ng/mL | | | | | | | |
| LOD | 0.010 | 0.044 | 0.062 | 0.054 | 0.078 | 0.020 | 0.040 |
| μg/g | | | | | | | |
| LLOQ | 0.03 | 0.15 | 0.22 | 0.18 | 0.26 | 0.066 | 0.13 |
| μg/g | | | | | | | |

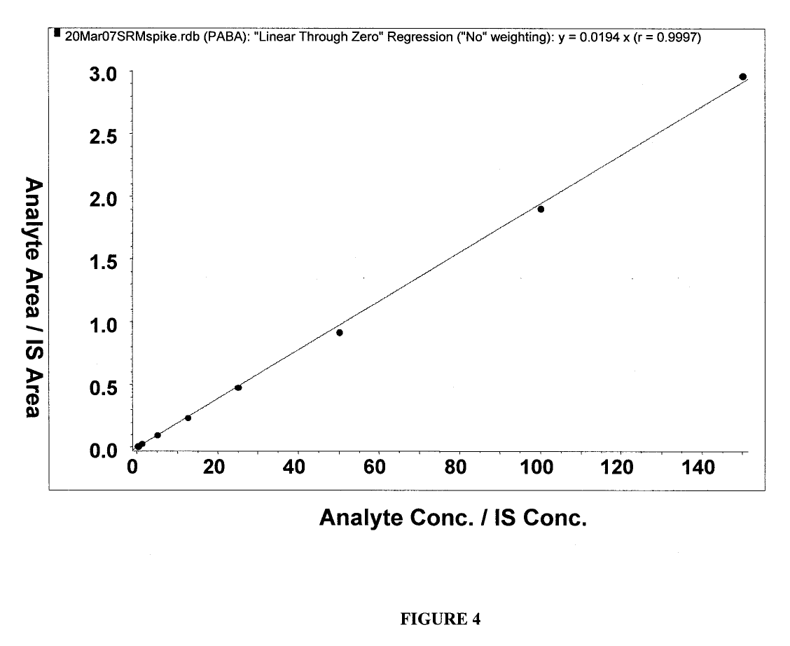

Linearity. The linearity for the analytes was good, with a coefficient of 0.9997 or higher, at the concentration ranges from 1 to 600 ng/mL for B₃mide and B₅, and 0.25 to 150 ng/mL for the other B vitamins measured. FIG. 4 shows a plot of analyte area over internal standard area versus analyte concentration over internal standard concentration. The linearity of calibration curves is shown in Table 9.

TABLE 9
Linearity of calibration curve.

| | B₁ | B₂ | B₃mide | B₃acid | B₅ | B₆ | PABA |
|---|---|---|---|---|---|---|---|
| Day | 0.9999 | 0.9999 | 0.9999 | 0.9999 | 1.0000 | 1.0000 | 1.0000 |
| 1 | | | | | | | |
| Day | 0.9999 | 0.9999 | 0.9999 | 0.9999 | 1.0000 | 0.9999 | 1.0000 |
| 2 | | | | | | | |
| Day | 0.9999 | 1.0000 | 1.0000 | 0.9997 | 1.0000 | 1.0000 | 0.9999 |
| 2 | | | | | | | |

These results demonstrate that the method is accurate and rapid for measurement of water soluble vitamins in infant formulas. The LC/MS/MS method is accurate and utilizes simple sample preparation, which allows a short analysis time and therefore provides the ability to analyze numerous samples more efficiently. In addition, multiple analytes can be measured in a single injection, allowing different chemical forms to be monitored. With the capability of measuring trace level of the analytes, it is expected that this LC/MS/MS method has potential application to analysis of other foods.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

What is claimed is:

1. A method for determining the level of B vitamins in a test sample, comprising:
(a) obtaining a water soluble vitamin fraction by extraction in acidic aqueous solution of a test sample, wherein said vitamin fraction comprises one or more B vitamins having at least a predetermined minimum concentration;
(b) adding a vitamin standard comprising one or more B vitamins of known quantity to a portion of said vitamin fraction of said test sample, wherein said vitamins in said vitamin standard comprise an isotope label and wherein said vitamin standard is added in an amount sufficient to determine the level of the corresponding vitamins in said test sample; and
(c) determining the level of one or more vitamins in said test sample corresponding to one or more of the vitamins in said vitamin standard using mass spectrometry.

2. The method of claim 1, wherein said vitamin standard comprises a B vitamin selected from vitamin B₁, vitamin B₂, vitamin B₃, vitamin B₅, vitamin B₆ and p-aminobenzoic acid (PABA).

3. The method of claim 1, wherein said vitamin fraction is obtained by extraction with an acidic alcohol solution.

4. The method of claim 3, wherein said alcohol is methanol.

5. The method of claim 1, wherein said isotope label comprises ²H, ¹³C or ¹⁵N.

6. The method of claim 1, wherein said test sample is a food product.

7. The method of claim 1, wherein said test sample is milk or a milk-derived product.

8. The method of claim 1, wherein said test sample is infant formula.
